(19) United States
(12) Patent Application Publication
(10) Pub. No.: US 2010/0062056 A1
(43) Pub. Date: Mar. 11, 2010

(54) ENCAPSULATED PICOPLATIN

(75) Inventors: Alistair J. Leigh, Seattle, WA (US); Christopher A. Procyshyn, Surey (CA); Angelica Phillips, Lynnwood, WA (US); Hazel B. Breitz, Seattle, WA (US)

Correspondence Address: SCHWEGMAN, LUNDBERG & WOESSNER, P.A., P.O. BOX 2938, MINNEAPOLIS, MN, 55402, US

(73) Assignee: Poniard Pharmaceuticals, Inc., Seattle, WA (US)

(21) Appl. No.: 12/536,311

(22) Filed: Aug. 5, 2009

Related U.S. Application Data
(63) Continuation of application No. PCT/US2008/001746, filed on Feb. 8, 2008.
(60) Provisional application No. 60/889,675, filed on Feb. 13, 2007.
(60) Provisional application No. 60/984,156, filed on Oct. 31, 2007.
(60) Provisional application No. 60/989,020, filed on Nov. 19, 2007.
(60) Provisional application No. 60/889,201, filed on Feb. 9, 2007.

(51) Int. Cl. A61K 31/555; A61K 9/48; A61P 35/00

(52) U.S. Cl. 424/451; 514/188

(57) ABSTRACT

The invention provides an encapsulated unit dosage form for picoplatin that is adapted for oral administration of the picoplatin containing a substantially dry powder with about 20 to 55 wt % picoplatin in the physical form of a picoplatin particulate wherein an average picoplatin particle diameter is less than about 10 microns. The picoplatin particles are dispersed within the powder of the formulation which includes a substantially water-soluble, water-dispersible, or water-absorbing carbohydrate and an effective amount of up to about 5 wt % of a lubricant.

### RELATED APPLICATIONS

This application is a continuation under 35 U.S.C. 111(a) of International Application No. PCT/US2008/001746 filed Feb. 8, 2008 and published in English as WO 2008/097658 on Aug. 14, 2008, which claims the benefit of provisional Application Ser. No. 60/889,675, filed Feb. 13, 2007 and to provisional Application Ser. No. 60/984,156, filed Oct. 31, 2007 and to provisional Application Ser. No. 60/989,020, filed Nov. 19, 2007 and to provisional Application Ser. No. 60/889,201, filed Feb. 9, 2007, which applications and publication are incorporated herein in their entireties.

### FIELD OF THE INVENTION

The field of the invention is encapsulated unit dosage forms for the anti-cancer organoplatinum drug picoplatin adapted for oral administration, processes of preparation of the unit dosage form, and methods of use of the unit dosage form.

### BACKGROUND

Picoplatin is a new-generation organoplatinum drug that has promise for treatment of various types of malignancies, including those that have developed resistance to earlier organoplatinum drugs such as cisplatin and carboplatin. Picoplatin has shown promise in the treatment of various kinds of cancer or tumor, including small cell lung cancer, colorectal cancer, and hormone-refractory prostate cancer.

Structurally, picoplatin is:

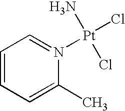

and is named cis-amminedichloro(2-methylpyridine)platinum(II), or alternatively [SP-4-3]-ammine(dichloro)(2-methylpyridine)platinum(II). The compound is a square planar complex of divalent platinum that is tetracoordinate and has three different ligand types. Two ligands are anionic, and two are neutral; therefore as the platinum in picoplatin carries a +2 charge, picoplatin is itself a neutral compound and no counterions need be present. The name “picoplatin” referring to the presence of α-picoline (2-methylpyridine) in the molecule is the United States Adopted Name (USAN), the British Approved Name (BAN), and the International Nonproprietary Name (INN) for this material. Picoplatin is also referred to in the literature as NX473, ZD0473, and AMD473, and is disclosed in U.S. Pat. Nos. 5,665,771, 6,518,428, and U.S. Ser. No. 10/276,503.

Tetracoordinate square planar platinum (II) are well known to be subject to oxidation to octahedral Pt(IV) complexes, such as with molecular chlorine. Also, it is well known that square planar platinum (II) complexes are subject to axial attack in ligand displacement reactions by various nucleophiles such as halides, amines, thio compounds, and under some conditions, water. Therefore, while picoplatin is relatively stable in pure form, in the absence of light, it can be subject to degradation under certain conditions, such as in the presence of nucleophilic molecular entities. See Advanced Inorganic Chemistry, F. Albert Cotton and Geoffrey Wilkinson, Second Revised Edition (1966) and later editions, Interscience Publishers. When administered to patients, picoplatin is believed to undergo transformation to some extent to two distinct aqua forms resulting from displacement of either of the chloride ligands. In addition to picoplatin, these cationic species (resulting from displacement of a chloride anion by neutral water) are also able to interact with cellular DNA to bring about cross-linking and eventual cell death. Picoplatin is also known to be unstable in the presence of certain metal oxides, such as iron oxide.

Picoplatin has previously been provided to patients in solution by intravenous (IV) administration. Picoplatin under standard conditions is a solid, and has only sparing solubility in water. The relatively low solubility of picoplatin in water (less than 1 mg/mL) necessitates that substantial volumes of liquid be delivered intravenously to provide a patient with total doses in the range of 100 mg and more (i.e., at a concentration of 0.5 mg/mL, some 200 mL of liquid must be introduced by IV infusion to provide a 100 mg dose). As typical human dosages for cancer patients can be on the order of several hundred milligrams per administration, and may be repeated every few weeks, substantial volumes of liquid must be delivered to the patient for each administration of the substance by the IV route. Intravenous administration is thus undesirable due to the need for needle insertion into a vein, and the relatively prolonged periods over which the patient must be immobile to allow for infusion of the relatively large volumes of the picoplatin solutions. Picoplatin is also known to be particularly susceptible to photo-decomposition when in solution, as in an IV dosage form. Picoplatin has been shown to be orally bioavailable in animals, but its low solubility in water, cytotoxicity and teratogenicity pose obstacles to the preparation of effective oral dosage forms. Therefore there is a need for effective dosage forms of picoplatin.

### SUMMARY OF THE INVENTION

The invention provides a solid unit dosage form for picoplatin, adapted for oral administration of the picoplatin to a mammal, such as a human afflicted with cancer. The oral picoplatin can be administered as a single agent or in combination with at least one other anti-cancer agent. The other anti-cancer agent is preferably a non-platinum anti-cancer agent that preferably is also administered orally.

The dosage form preferably comprises a substantially water-soluble capsule shell, the shell enclosing a formulation comprising a substantially dry, finely particulate material comprising, in admixture, about 10 to 60 wt % picoplatin, wherein the picoplatin is, in physical form, particulates of less than about 10 microns average particle diameter, in admixture with a substantially water-soluble, water-dispersible, or water-absorbing carbohydrate and an effective amount of up to about 5 wt % of a lubricant (or “glidant”). The capsule shell is preferably composed of a biodegradable and/or digestible material, such as hard or soft gelatin, PVA, polylactides, polyglycolic acids, and the like. The picoplatin preferably is a particulate having an average particle diameter of 1-5 microns. The picoplatin particulate can be micronized, for example by jet-milling, or can be a microcrystalline material, such as can be prepared by precipitation, or can be a particulate formed by a lyophilization process, or any combination of the three processes. The picoplatin particulate can be dispersed within substantially every particle of the powder of the formulation.

The invention also provides a process for preparing an encapsulated unit dosage form for picoplatin, adapted for oral administration of the picoplatin, comprising preparing a formulation comprising a substantially dry powder comprising about 20 to 55 wt % picoplatin wherein the picoplatin is particulates of less than about 10 microns average particle diameter, a substantially water-soluble, water-dispersible, or water-absorbing carbohydrate, and an effective amount of up to about 5 wt % of a lubricant; then, enclosing the formulation within a substantially water-soluble capsule shell.

Optionally, the formulation also comprises an effective amount of a dispersing agent, as discussed below.

The invention also provides a method of treating cancer in a mammal afflicted therewith, comprising orally administering a solid or liquid dosage form, including the encapsulated unit dosage form of the invention or the encapsulated unit dosage form prepared by the process of the invention, in a total dosage, at a frequency, and over a period of time adequate to provide a beneficial effect to the mammal.

Other solid dosage forms useful in the invention, preferably with drug-compatible coatings, include pills, tablets, sachets and the like. The picoplatin unit dosage form may be an encapsulated liquid or gelled composition; a coated tablet, a depot delivery system or an ingestible liquid composition.

Typically, total picoplatin doses are about 1 μg-400 mg per administration. The dosage form is administered to the mammal at intervals of about every day for at least two days, e.g., for 2-28 days at intervals of every 1-6 weeks. The picoplatin administration can be accompanied by anti-emetic therapy, such as use of a corticosteroid such as dexamethasone, a 5-HT3 inhibitor such as palonosetron or ondansetron, a tranquilizer such as lorazepam, or any combination thereof. The amount of picoplatin in the present dosage forms can be readily modified, e.g., so that using small amounts can be delivered in a unit dosage form, e.g., about 0.5-100 μg, or less.

The present invention thus provides a unit dosage form comprising encapsulated picoplatin that is at least about 10%, 20%, 30%, 40% or 50% bioavailable to a mammal upon ingestion of the dosage form by a mammal, e.g., a human, or domestic animal, such as a dog, cat, bovine, equine and the like.

The picoplatin oral dosage form of the invention has up to about 40-50 percent bioavailability, following oral ingestion by a human. More specifically, the picoplatin oral dosage form of the invention has about 10-50%, or about 20-45%, or about 30-40% bioavailability in humans following oral ingestion. Alternatively, the picoplatin oral dosage form of the invention has about 30-50%, or about 40-50% bioavailability in mammals following oral ingestion.

In another embodiment, the present invention provides a method for treating cancer providing orally administering to a mammal, such as human, afflicted with cancer an amount of picoplatin effective to maximize, or to approach maximization, of the concentration of picoplatin in the blood of the mammal, wherein the administration is carried out at least once a day for about one to four consecutive days. Preferably the administration is carried out by consecutive daily oral ingestion by the mammal, such as a human cancer patient, of one or more solid unit dosage forms comprising an amount of picoplatin, so as to provide the effective amount of picoplatin, as described herein. The mammal is then not dosed for a period effective to eliminate substantially all, e.g., about 90-100% of the picoplatin from the blood of the mammal, and/or to permit recovery from any immunosuppressant side effects or other side effects, if any, caused by administration of the picoplatin. The recovery period can be about 1-2 weeks, or longer, as needed. The cycles of daily administration for a plurality of days, each followed by the recovery period may be repeated as needed. The amount of picoplatin effective to maximize or to saturate the blood plasma level can be about 300-500 mg per day, for a human cancer patient, given in a single dose each day or in multiple daily doses. This regimen rapidly “spikes” or maximizes the circulating levels of picoplatin, to achieve the maximum tolerated levels, in order to aggressively treat the cancer.

In another embodiment of the invention, the present invention provides a method for treating cancer providing orally administering to a mammal, such as a human afflicted with cancer, an amount of picoplatin effective to provide a substantially constant level of picoplatin in the blood of the mammal for an extended period of time. Such levels can be relatively low compared to saturation or to the maximum “loading” level. This can be accomplished, for example, by daily oral administration of dosages of about 1.0 μg-10 mg of picoplatin, for up to about 3-5 weeks. The mammal is then not dosed for a recovery period effective to substantially eliminate the picoplatin from circulation, e.g., for about 1-2 weeks. This period can permit recovery of the immune system of the mammal from any immunosuppressant effects or other side effects, if any, due to the picoplatin. The cycles of the relatively prolonged, low-dose administration of picoplatin, followed by a recovery period may be repeated, e.g., for 2-10 cycles, as needed. This dosage regimen provides relatively low but constant levels of circulating picoplatin to provide continuous contact of therapeutic amounts of picoplatin with cancer cells.

In some cases, the daily dosing can be continued for up to 1-2 years with no prolonged interruption of dosing. This “metronomic dosing” can be particularly useful in adjuvant or metastatic settings, and/or when using with second anti-cancer agents. A prolonged interruption is about 2-3 weeks, in standard therapy.

The present invention provides the use of a unit dosage form of picoplatin adapted for oral administration, preferably with a unit dosage form of a non-platinum anti-cancer agent, that is preferably adapted for oral administration, to treat cancer.

### DETAILED DESCRIPTION OF THE INVENTION

### Definitions

“Picoplatin” refers to cis-amminedichloro(2-methylpyridine)platinum(II), or [SP-4-3]-ammine(dichloro)(2-methylpyridine)platinum(II) as the drug is also termed, the structure of which is shown above. It is a compound belonging to the general class of transition metal complexes, in this case a complex of the third-row transition element platinum, the platinum being in the +2 oxidation state.

A “unit dosage form” or an “encapsulated unit dosage form” as used herein refers to a physical dosage form that is adapted for oral administration, e.g., ingestion, wherein the form provides a preselected dose per subject, adapted to provide for a complete and release of the drug, which may be rapid, controlled or prolonged release, in vivo after administration of the dosage form.

In accord with the present invention, the solid particulate formulation including the picoplatin and excipients are contained within a substantially water-soluble shell, typically a gelatin or hydroxypropyl methyl cellulose (HPMC) capsule, which contains the formulation during storage and oral ingestion. By “substantially water-soluble” is meant that the capsule shell is sufficiently water-soluble to allow the shell to dissolve or rupture in the gastro-intestinal (GI) tract of the mammal, so that the active ingredient of the formulation, picoplatin, can be absorbed into the mammal's bloodstream through the mucosa of the GI tract. Thus, dissolution of the substantially water-soluble shell takes place within the period of time of a typical residence of an ingested substance within the GI tract, for example, within a period of time of several hours, preferably within a period of time of less than about 30 minutes, more preferably within a few minutes after ingestion of the capsule by the patient. However, although rapid dissolution is preferred, the tablet can be further coated or otherwise designed to permit controlled or prolonged release of the picoplatin if desired.

A “capsule band” or “banding” as the terms are used herein refers to a substantially water-soluble band that encloses the capsule, serving to join the two halves of the capsule together such that the capsule cannot readily be pulled apart by the administrators or the subjects to release the encapsulated picoplatin formulation.

The use of an encapsulated dosage form for picoplatin is advantageous in terms of protecting care providers, patients, persons involved in the packaging of the dosage forms, and other persons who may come in contact with the capsules, from exposure to the cytotoxic picoplatin. Picoplatin in powder form may be inhaled as a dust, inadvertently orally ingested, or absorbed through breaches in the skin. Tablets, which are composed of a compacted powder, are susceptible to abrasion and the formation of dusts from the outer layer of the tablet, even if coated. Handling of tablets can result in deposition of the cytotoxic picoplatin on the skin, and its subsequent ingestion. In contrast, a capsule, composed of a solid material like gelatin or HPMC, forms a physical barrier to the solid picoplatin that the capsule encloses. This provides for safer handling for persons handling the dosage forms, for examples, pharmacists and nurses. For an additional level of safety, capsules that include two halves can be banded after filling. The band covers the seam between the two halves of the capsule, thus preventing the accidental or purposeful separation of the halves and resulting release of the cytotoxic picoplatin powder. For example, a gelatin band can be affixed to the capsule containing the formulation in such a way as to irreversibly seal the capsule shut.

A “light-attenuating” capsule shell, as the term is used herein, refers to a capsule shell that is adapted or treated so as to attenuate the intensity of light transmitted by the capsule shell. A shell may be light-attenuating without completely blocking or reflecting all incident light within the meaning herein. An “opaque” capsule shell is a shell that substantially completely blocks or reflects all incident light.

A process can be carried out under “subdued illumination,” as defined herein, when light intensities lower than the light intensities commonly used in manufacturing facilities, i.e., illuminations of an intensity sufficient to read written text, are used. Subdued light can also refer to light of spectral distribution known as “safe-light,” that is, light consisting predominantly of frequencies in the yellow to red range of the spectrum, where the picoplatin light absorption is less intense on a molar basis. Due to the potential light sensitivity of picoplatin, subdued illumination during practice of the process of the invention, along with the use of a substantially light-attenuating capsule shell, can serve to enhance the stability and preserve high purity of the picoplatin in the present dosage form.

A “formulation” as the term is used herein refers to a powder that comprises picoplatin, the picoplatin in physical being a particulate of less than about 10 microns average particle size, the formulation further including a carbohydrate and a lubricant, as the terms are defined herein. The formulation may also include other ingredients, such as a dispersant/disintegrant, an antioxidant, a buffer, a colorant, and the like. The formulation is enclosed by the capsule shell to provide the unit dosage form of the invention.

A “carbohydrate” as the term is used herein includes carbohydrates useful as fillers or as bulking agents in pharmaceutical compositions, including a monomeric, dimeric, oligomeric or polymeric sugar derivative, such as glucose, fructose, lactose, sucrose, ribose, hemicelluloses, celluloses, modified celluloses (cellulose ethers, etc.), and the like. A carbohydrate molecule comprises carbon, hydrogen and oxygen, in an approximate molar ratio of 1:2:1. However, molecules deviating from this formula, such as deoxysugars and their oligomers/polymers, are also included within the term “carbohydrate” as used herein, provided sufficient hydroxyl groups are present to confer water-solubility or water-absorbability upon the substance. A carbohydrate may also contain other elements such as nitrogen (e.g., aminosugars), sulfur (e.g., sugar sulfonic acids), and phosphorus (e.g., sugar phosphates), without departing from the principles of the invention.

By a “substantially water-soluble” carbohydrate is meant that the carbohydrate is sufficiently water-soluble to allow it to dissolve in the aqueous environment of the gastrointestinal (GI) tract within a few hours, preferably within a few minutes. An example of a substantially water-soluble carbohydrate is a monosaccharide, for example glucose.

By a “substantially water-dispersible carbohydrate” is meant a carbohydrate that, while it may not totally dissolve in water, is nevertheless of sufficient hydrophilic nature that it freely disperses in water.

By a “substantially water-absorbing” carbohydrate is meant that the carbohydrate, although it does not completely or even to any significant degree dissolve in water, it nevertheless takes up, adsorbs, or absorbs water within its physical structure. For example, cellulose, such as microcrystalline cellulose, does not dissolve in water, but it becomes hydrated in the presence of water, absorbing several times its weight in water. This absorption of water by, for example, cellulose, can assist in the dissolution of the picoplatin; it is believed that this absorption of water by the water-absorbing carbohydrate acts to assist in the dissolution of the picoplatin within the GI tract, by holding water molecules within close physical proximity to the surfaces of the finely particulate picoplatin.

By a “substantially dry” material is meant a solid substance to which no exogenous water has been added and which has a relatively low wt % of contained water, typically less than about 5 wt %, preferably less than about 1-3 wt % of water, more preferably less than about 1 wt % of water. A substantially dry material need not be absolutely anhydrous within the meaning assigned herein, but the amount of residual water present in the material is limited. For example, lactose monohydrate, which includes 5 wt % water, can be used as a carbohydrate in the dosage form.

By a “powder” is meant a material in the physical form of a solid that is divided into relatively fine particles. A powder can be a milled powder. Such powders can be made by grinding coarser powders to the desired fineness. A preferred method of forming a micronized powder is by jet milling. The powder material that is encapsulated contains the picoplatin particulate, a fine powder of less than 10 microns average particle diameter, in combination with, or incorporated within, coarser powders such as carbohydrates, which can be of sufficient fineness to pass a 20-mesh or a 30-mesh screen, but which need not be of less than 10 microns average particle diameter.

By a “particulate,” in the context of the physical form of solid picoplatin disclosed herein, is meant a very fine powder wherein the average picoplatin particle diameter is less than 10 microns, preferably less than 7 microns, most preferably wherein at least about 90% of a sample of the particulate material is composed of individual particles each having a diameter of less than about 5 microns. The finely particulate nature of the picoplatin aids in its rapid and complete dissolution in the patient's GI tract. The picoplatin particulate can be a micronized material, a microcrystalline material, a lyophilized material, or any combination thereof.

A “micronized” material is a particulate wherein the majority of the particles making up the powder have a particle diameter of about 10 microns or less. Preferably, the average particle diameter is about 5 microns or less. Particle diameters can range down to about 1 micron or less without departing from the principles of the invention. A micronized solid can be crystalline or amorphous.

A “microcrystalline” material is a fine particulate wherein the solid is in crystalline form, the crystals being predominantly of the specified dimensions. A microcrystalline material can be prepared by precipitation of the material from a solvent, such as by addition of a second liquid material in which the material is insoluble.

A “lyophilized” material is a solid that has been obtained by a step of lyophilization of a solution of the material. Lyophilization, as is well known, involves the vacuum sublimation of a solvent such as water, or other compatible solvent(s), from a frozen solution of the material, such that once the solvent is completely removed, a finely powdered solid material remains.

By the term “cellulose” is meant herein a polymeric carbohydrate material made up mostly of a linear polymer of β(1-4)-linked D-glucose units. Cellulose is typically derived from a natural source such as wood pulp, cotton, or bacteria. Cellulose may be ground or comminuted to create a finely particulate material. Alternatively, microcrystalline cellulose, such as is sold under the trademark Avicel®, can be used. For example, the Avicel® can be Avicel PH101®. By microcrystalline cellulose is meant a cellulose which has been subjected to partial acid hydrolysis, which serves to predominantly hydrolyze the amorphous regions of a sample of cellulose, leaving the more crystalline domains intact. Microcrystalline cellulose takes the physical form of a fine powder.

The term “modified cellulose” as used herein refers to a chemically or biologically modified cellulose. For example, sodium carboxymethyl cellulose, that is, cellulose that bears pendant carboxymethyl groups as sodium salts, as is well known in the art, is a modified cellulose within the meaning herein. Likewise, methyl cellulose, ethyl cellulose, hydroxyethyl cellulose, hydroxypropyl cellulose, and hydroxypropyl methyl cellulose (HPMC) are modified celluloses within the meaning assigned herein. A cross-linked sodium carboxymethyl cellulose, also known as “croscarmellose sodium,” is a cross-linked modified cellulose within the meaning herein. Croscarmellose sodium is a dispersant/disintegrant within the meaning of the term herein.

A “lubricant” or “glidant” within the meaning herein is a substance that serves to coat the surface of particles and reduce the friction of inter-particle movement, such as during powder handling operations, for example, when filling capsules. Reducing the friction serves to reduce static electricity buildup and particle clumping or aggregation, for example during the milling, powder handling, and capsule filling processes typically used to produce the unit dosage form of the invention or a unit dosage form produced by the method of the invention.

A “dispersant” or “disintegrant” is a substance that is a component of a formulation of the invention that aids in the dispersion of the finely particulate formulation of the invention upon exposure to an aqueous medium, for example within the GI tract of a patient to whom the unit dosage form of the invention has been administered. It is believed that dispersants act to increase the solvation of the surfaces of solid particles within the aqueous medium, thereby reducing particle-particle adhesion and clumping while aiding in dissolution of the solid through improved surface wetting. Examples of dispersant include croscarmellose sodium and povidone. Povidone, also known as poly(vinylpyrrolidone), is a polymeric material bearing multiple pyrrolidone units along a poly(vinyl) backbone.

The present invention provides a unit dosage form for picoplatin, adapted for oral administration of the picoplatin, comprising a substantially water-soluble capsule shell, the capsule shell enclosing a formulation comprising a substantially dry powder comprising about 10 to 60 wt % picoplatin, preferable about 15-40 wt %, wherein the picoplatin is in physical form a particulate of less than about 10 microns average particle diameter, about 40-80 wt % of a substantially water-soluble, water-dispersible, or water-absorbing carbohydrate, and an effective amount of up to about 5 wt % of a lubricant.

The unit dosage form, which is adapted for oral administration of picoplatin, includes a capsule shell, which encloses the formulation comprising the picoplatin, wherein the capsule shell is formed of a substantially water-soluble material. The capsule shell is sufficiently water-soluble to allow the shell to dissolve or rupture in the gastro-intestinal (GI) tract of the mammal, so that the formulation is released for dissolution and absorption into the blood stream through the mucosa of the GI tract.

Whereas any plastic non-toxic water-soluble material that is suitable for consumption can be used to form the capsule shall, the capsule shell material is preferably made of gelatin, such as a hard gelatin, as is well known in the art. “Gelatin,” as the term is used herein, is a collagen-derived material that is about 98-99% protein by dry weight. The approximate amino acid composition of gelatin is: glycine 21%, proline 12%, hydroxyproline 12%, glutamate 10%, alanine 9%, arginine 8%, aspartate 6%, lysine 4%, serine 4%, leucine 3%, valine 2%, phenylalanine 2%, threonine 2%, isoleucine 1%, hydroxylysine 1%, methionine and histidine<1%, and tyrosine<0.5%. Gelatin capsule shells, as are well-known in the art, can be adapted to rapidly dissolve in the GI tract. Specifically, a size 3 gelatin capsule can be used as a shell. The capsule shell can also be composed of gelatin/PEG (a gelatin derivatized with polyethylene glycol) or of hydroxypropyl methyl cellulose (“HPMC”). Such two-piece HPMC capsules include Quali-V® capsules, available from Shionogi Qualicaps.

The capsule shell of the invention can be banded, that is, sealed with a band of a preferably water-soluble material that serves to cover the seam between the two halves of the capsule shell and to hold the two halves of the capsule shell together such that the capsule cannot be readily pulled apart to release the cytotoxic picoplatin powder.

The capsule shells of the invention can be adapted to attenuate incident light as may fall on the unit dosage form. Due to the known instability of picoplatin to light, attenuation of incident light serves to maintain the purity of the picoplatin contained within the capsular dosage form. The capsule shell can attenuate incident light to a significant degree, preferably at least by about 50% at typical room illumination levels, or at least by about 75%, or at least by about 90%, or at least by about 95%. This can be accomplished by incorporation into the shell of a suitable opaquifying agent, for example, a metal oxide such as TiO₂ or Fe₂O₃. The opaquifying agent serves to attenuate incident light by absorbing or reflecting the light. TiO₂, which is white in color and has a high albedo, reflects light across the visible portion of the spectrum with high efficiency. The opaquifying agent can be incorporated into the capsule shell, for example by inclusion in gelatin, or can be a component of a formulation that is coated onto the capsule shell, preferably on the exterior of the shell to avoid contact of the metal oxide with the picoplatin. This serves to protect the contents of the capsule from light, and assists in maintaining a high level of purity of the contained picoplatin.

The capsule shell of the invention contains a formulation comprising a powder comprising picoplatin in physical form a particulate of less than about 10 microns average particle diameter in admixture with a carbohydrate and a lubricant. Multiple types of carbohydrate, lubricant, or both, can be present. Additional ingredients can also be present in the formulation, such as a dispersant, which can serve to disperse the particles of the picoplatin-containing powder in the patient's GI tract. Other ingredients that can be present include stabilizers such as anti-oxidants, buffers, colorants, or other medicaments, including anti-cancer drugs.

As mentioned above, picoplatin is a tetracoordinate platinum(II) complex, and such complexes are known to possess certain instabilities, that the unit dosage form of the invention is adapted to avoid or minimize. For example, tetracoordinate platinum(II) complexes, as described above, are susceptible to addition of molecular chlorine. Molecular chlorine can be formed in situ when chloride (a halide) and an oxidizing reagent (such as atmospheric molecular oxygen) are present. Thus, chlorides are preferably excluded from the formulation. Solid oxidizing agents that can be used, for example, as microbiocides, including chlorite, chlorine dioxide and povidone iodine, are preferably excluded from the formulation. Also, since compounds comprising moieties including ═NH, —NH₂, and —SH, as can be found in various excipients such as dispersant/disintegrants, can react with tetracoordinate platinum(II) complexes like picoplatin, either in situ or in vivo, the dosage form preferably does not include any such compounds.

Picoplatin can be decomposed by contact with metal oxides, such as iron oxide, titanium dioxide, copper oxides, iron oxides, zinc oxide, and the like, that are used in coating tablets. For this reason, use of an encapsulated oral dosage form of picoplatin may be advantageous relative to a tablet oral dosage form in that, in a capsular form, no additional intimate or continuous coating of the picoplatin formulation is required to protect the picoplatin.

In the encapsulated formulation, any metal oxides such as titanium oxide that may be incorporated into the capsule, for example as opaquifying agents, do not come into physical contact with the picoplatin. As metal oxides such as titanium oxide are well adapted to be opaquifying agents, it is preferred to use them to block exposure of the picoplatin to light, and by incorporating such an opaquifying agent into the capsule material, for example hard gelatin or HPMC, or by coating the exterior of the capsule with a material containing a metal oxide opaquifying agent, light attenuation is achieved without undesired metal oxide induced picoplatin decomposition.

While the solid formulation as contained within the shell may be only moderately susceptible to picoplatin degradation in the presence of reactive functional groups such as amino groups, due to the relatively unreactive nature of solid materials, during processes such as in the compounding of the formulation, and particularly during the process of capsule rupture and dissolution in the stomach, the absence of reactive ingredients can assist in maintaining picoplatin purity. In the microenvironment that exists as the solid formulation is first released into the stomach acid after capsule dissolution or rupture, local high concentrations of formulation ingredients exist in close physical proximity to the surfaces of the dissolving picoplatin particulates. It may take several minutes, if not longer, for these small particulates with their high surface area to pass completely into solution, and during that time the ingredients of the formulation other than the picoplatin are likewise dissolving, and are present in solution in high local concentrations adjacent to the dissolving picoplatin particles. The absence of picoplatin-reactive functional groups on substances that can exist in locally high concentrations in these stomach microenvironments is therefore advantageous.

The picoplatin that is contained in the powder of the formulation is in physical form a particulate of an average particle diameter of less than about 10 microns. The picoplatin particulate can be a micronized material, a microcrystalline material, a lyophilized material, or any combination thereof. The picoplatin can be milled or micronized by jet milling, or by any other process that can provide micronized powders of suitably small average particle diameters. Micronized picoplatin, due to the favorable surface area to mass ratio that results from the presence of fine particles, aids in the rapid and complete dissolution of an effective amount of the picoplatin in the patient's GI tract after administration of the dosage form. Micronized picoplatin can be composed of crystalline or amorphous solid picoplatin.

The picoplatin particulate can also be a microcrystalline solid, wherein the powder is composed of crystals of appropriately small physical dimension. Microcrystalline materials can be formed, as is known in the art, by precipitation of a solid from a solution by addition of a liquid in which the material is insoluble, for example with high shear or agitation.

The picoplatin particulate can also be a lyophilized powder, such as is formed by lyophilization of a solution of the picoplatin. The picoplatin particulate can also have been formed by any combination of the above-listed methods of forming fine particulates; for example, a microcrystalline material can be micronized such as by jet milling to reduce particle size, or a material that has been recovered from an aqueous solution by lyophilization can be micronized, and so forth.

The admixture of the picoplatin, the carbohydrate, the lubricant, and any other ingredients that may be present is also a powder, but is not as fine a powder as the picoplatin particulate. The formulation can be a mixture of picoplatin particulates and particles of the other ingredients, or, preferably, the particles making up the powder of the formulation can have incorporated within substantially every one of them a plurality of picoplatin particulates dispersed in admixture with the other components such as the carbohydrate. The powder of the formulation can be fine enough to pass, for example, a 20-mesh screen or a 30-mesh screen. The mixed powder can be a milled powder. It is preferred that the admixture be an intimate admixture, where picoplatin particulates are closely mixed with the additional ingredients of the formulation, as the greater the surface area of the component picoplatin particles, and the more intimately these picoplatin particles are mixed with the carbohydrate, and with the optional dispersant or disintegrant, the more rapidly and completely the picoplatin will dissolve or disperse after administration of the capsule to the patient. Rapid and complete dissolution of the picoplatin is desirable in terms of providing a maximally effective treatment to the patient.

The powder that is enclosed by the capsule shell is in a substantially dry form; the water content of ingredients such as carbohydrates and dispersants is controlled to minimize the wt % of water in the formulation. Water, under some conditions, can react with picoplatin, resulting in decomposition. Therefore, the water content of the dosage form is preferably limited to less than about 5 wt %, preferably less than about 1-3 wt %, and more preferably to less than about 1 wt % of the composition. It is understood that certain carbohydrates, for example lactose, may exist in the form of a hydrate, such as a monohydrate, and such hydrates may be used without departing from the principles of the invention, but exogenous water is preferably excluded as much as is practicable.

The picoplatin, which makes up at least about 10-20 wt % of the formulation and can make up to about 55-60 wt % of the formulation, is preferably anhydrous, and is handled under conditions during the formulation processes to maintain its dry state.

Suitable carbohydrates can be selected from a group consisting of a monosaccharide, a disaccharide, a sugar alcohol, a cellulose, a modified cellulose and mixtures thereof. Carbohydrates are water-soluble, water-dispersible, or are water-absorbing, that is, the fillers either dissolve completely in water, freely disperse in water, or are sufficiently hydrophilic to absorb substantial amounts of water within their structure. For example, fructose is water-soluble, certain hemicelluloses are water-dispersible, and cellulose is water-absorbing. More than one carbohydrate can be present in the dosage form. The total carbohydrate is preferable present at about 40-90 wt % of the formulation.

An example of a monosaccharide is fructose. Other examples include without limitation glucose, xylose, mannose, galactose, ribose, and the like. Examples of a disaccharide include lactose and sucrose.

Examples of sugar alcohols include sorbitol, ribitol, mannitol and xylitol. An example of a hemicellulose is a wood-derived, alkali-soluble hemicellulose.

An example of a cellulose is microcrystalline cellulose. Another cellulose is a finely ground or comminuted cellulose, such as a high grade wood pulp cellulose that has been ground to a powder form.

An example of a modified cellulose is sodium carboxymethyl cellulose. Other examples include without limitation methyl cellulose, ethyl cellulose, hydroxyethyl cellulose, hydroxypropyl cellulose, and hydroxypropyl methyl cellulose. Some examples of modified cellulose are water soluble, whereas others are water-dispersible or water-absorbing.

The formulation of the invention includes a lubricant in an effective amount. A lubricant, for example the salt of a fatty acid, more specifically magnesium stearate, can serve as a processing aid in handling the powder of the formulation, in particular the sub-10 micron picoplatin powder, by assisting in avoidance of particle clumping, such as during milling and encapsulation operations. A lubricant can be present at up to about 5 wt % of the formulation.

A dispersant, which serves to enhance the dispersal of the powder of the formulation in an aqueous medium, such as in the GI tract of a patient, facilitates the rapid dissolution of the formulation after rupture or dissolution of the capsule shell. The dispersant tends to inhibit aggregation or clumping of the particles when they first encounter the aqueous medium, thus helping to preserve the favorable surface area to mass ratio of the micronized picoplatin powder. An example of a dispersant is cross-linked sodium carboxymethyl cellulose, also known as croscarmellose. Another example is povidone, also known as polyvidone, poly(vinylpyrrolidone), or PVP. The formulation can comprise about 5-10 wt % of the dispersant. More than one dispersant can be present in the formulation.

The formulation can include other ingredients, but preferably does not include oxidants, metal oxides, or compounds comprising halo, ═NH, —NH₂, or —SH moieties. For example, anti-oxidants can be included. Colorants, such as food dyes, can be included.

Thus the ratio of picoplatin to carbohydrate filler to dispersant (if present) to glidant is about 1:1.5-3.0:0.1-0.3:0.25-0.1. In one embodiment, the unit dosage form of the invention comprises about 200 mg of the formulation of the finely particulate material, comprising about 50 mg of micronized picoplatin, about 116 mg of lactose monohydrate, about 20 mg of microcrystalline cellulose, about 8 mg of croscarmellose sodium, about 4 mg of povidone, and about 2 mg of magnesium stearate, as an admixture, contained in a gelatin capsule. Preferably, the powder contains about 25 wt-% picoplatin, about 68 wt-% of a mixture of lactose and microcrystalline cellulose, about 6 wt-% croscarmellose sodium and povidone and about 1 wt-% magnesium stearate. The gelatin capsule is preferably opaquified, as by incorporation of TiO₂ into the gelatin or cellulosic shell of the capsule or by coating the exterior of the gelatin capsule with a material that includes TiO₂.

The invention further provides a process for preparing an encapsulated unit dosage form for picoplatin, the unit dosage form being adapted for oral administration of the picoplatin, the process comprising preparing a formulation comprising a substantially dry, powder comprising picoplatin, wherein the picoplatin is in physical form a particulate of less than about 10 microns average particle diameter, a substantially water-soluble, water-dispersible, or water-absorbing carbohydrate, and an effective amount up to about 5 wt % of a lubricant; then, disposing the formulation within a substantially water-soluble capsule shell.

The constituent materials used in the process of the invention are as described above for the unit dosage form of the invention. The process of the invention comprises preparing the powder of the formulation that is substantially dry, and then filling the capsule shell with the material. The capsule shell is water-soluble and can be light-attenuating, as is described above.

For example, lactose monohydrate, microcrystalline cellulose, and a lubricant such as magnesium stearate can each be ground to pass a 20-mesh screen, then can be blended with the picoplatin particulate together in a granulator. The picoplatin particulate can be prepared earlier by a jet milling process, or can be microcrystalline, or can be lyophilized, or any combination of these processes that provides particulates of the requisite dimensions. A dispersant, for example povidone, such as in the form of a powder that passes a 20-mesh screen, can be added to the mixture in the granulator. Mixing of the solids then can take place using high-shear granulation, so as to form an admixture of the component materials. The picoplatin can be premixed with the carbohydrate, for example lactose, prior to mixing with additional ingredients. This can serve to reduce the amount of static electricity buildup on the picoplatin particulates. The admixture of the sparingly water-soluble picoplatin (having a solubility of about 1 mg/mL, or about 0.1%, in water) with the water-soluble, water-dispersible or water-absorbing carbohydrate, the lubricant, and optionally with the dispersant, serves to enhance rapid and substantially complete dissolution of an effective amount of the picoplatin in the patient's GI tract.

Following the milling and mixing processes, the formulation can be dried, for example, spread in a thin layer on a tray, which is then held under drying conditions. For example, the powder on the tray can be warmed to a moderate temperature, such as about 40-80° C., and held under a partial vacuum or in the presence of a drying agent, for example, P₂O₅. Residual water can be controlled such that the water content is less than 5 wt %, more preferably less than 1-3 wt %, even more preferably less than 1 wt %, of the solid mixture.

Following drying, additional milling can take place. The bulk material can be sifted through the screen, if desired, to remove any larger particles that may be present. For example, a predominant portion of a sample of a finely particulate material can pass through a 20-mesh screen. Preferably, the bulk of the powder can pass through a 30-mesh screen.

The powder of the formulation can be kept substantially dry through the use of suitable engineering techniques and controls, such as storage under controlled atmosphere, interjection of suitable drying steps into the process for preparation, and storage in the absence of atmospheric moisture. The powder can also be handled under subdued light, in order to minimize the amount of photolytic decomposition of picoplatin, which is well known to be light-unstable. The control of incident light can be carried out by suitable engineering controls, such as processing the material in opaque vessels, conveying it and drying it under cover or in the dark, or the use of safe-lights such as can be used for photographic processing. It is desirable to minimize incident light in carrying out the inventive process.

After the final mixing, drying, and screening steps of the process are carried out, the powder of the formulation is enclosed within the capsule shells, such as by techniques well known in the art. Again, it is preferred that the capsule filling and storage operations be carried out under subdued light and substantially dry conditions.

The invention provides one or more of dosage forms packaged with instruction materials regarding administration of the dosage form, or with instruction materials that comprise labeling means, e.g., labels, tags, CDs, DVDs, cassette tapes and the like, describing a use of the dosage form that has been approved by a government regulatory agency.

The dosage form can include means that provides identifying information useful to a care provider, such as a physician or a nurse, that can include the identity, concentration, expiration date. This can serve to avoid medical mistakes and to provide an additional level of assurance to the care provider and to the patient that the correct medication is being administered. The identifying information can be in a non-visual form so that it can be detected in low light, for example, by textural features of the capsule, raised letters signifying picoplatin and the dosage, and the like. Alternatively, the capsule can be colored in a manner that conveys dosing information or to identify the contents. For example, if a treatment session will use three capsules, the capsules can be coded, such as with different colors, to indicate to the care provider the relative position of a given capsule in the treatment sequence, first, second or third. This serves to avoid medical mistakes such as over- or under-dosing as could occur if the care provider loses count of the capsules administered to a patient in a treatment session.

As a light-sensitive compound, picoplatin and its solutions are protected from light exposure, for example, by packaging in opaque materials. Thus, dosage forms of the present invention can be shielded from light by secondary packaging that minimizes exposure to visible light.

The unit dosage form of the invention, or the unit dosage form prepared by the method of the invention, can have about a ±10% spread in the actual amount of contained picoplatin relative to the nominal composition. For example, a unit dosage form with a nominal 200 mg weight containing a nominal 50 mg of picoplatin, can have about 45 to 55 mg of picoplatin as measured for that individual sample. The unit dosage form of the invention has low and limited amounts of various impurities; for example it should contain no more than about 1% of each of several possible residual impurities from the manufacture or storage of the picoplatin, such as picoline, potassium tetrachloroplatinate, trichloropicoline platinate or trichloroaminoplatinate.

Other useful oral unit dosage forms include the coated tablets disclosed in Leigh et al., U.S. provisional application Ser. No. 60/889,171, filed Feb. 9, 2007, and A. Chen, U.S. provisional application Ser. No. 60/950,033, filed Jul. 16, 2007, which are incorporated herein by reference.

The invention also provides a method of treating cancer in a human afflicted therewith, comprising orally administering the unit dosage form of the invention or the oral unit dosage form prepared by the method of the invention, or a plurality thereof, in a total dosage, at a frequency, and over a period of time adequate to provide a beneficial effect to the mammal. The method can also comprise the administration, preferably the oral administration, of a second non-platinum anti-cancer agent, which is preferably administered orally.

It is well known that picoplatin can be active against tumors that possess, or have developed, resistance to “first-generation” or “second generation” organoplatinum anti-cancer drugs such as cisplatin and carboplatin. For example, the oral dosage form of the invention or prepared by the process of the invention can be used to treat patients with hematological and non-hematological malignancies, particularly non-hematological malignancies, such as patients with solid malignant tumors, in particular, those patients whose solid tumors are cisplatin, oxaliplatin, or carboplatin refractory. Specific types of solid malignancies that can be treated with the oral dosage form of picoplatin of the invention, or with a picoplatin oral dosage form prepared by the process of the invention, include without limitation, lung cancer, including small cell lung cancer, non small cell lung cancer, head and neck cancer, GI/stomach cancer, skin cancer, ovarian cancer, kidney cancer, bladder cancer, mesothelioma, prostate cancer, including hormone-refractory prostate cancer, cervical/uterine cancer, liver cancer, testicular cancer, pancreatic cancer, colorectal cancer, sarcomas, breast cancer, carcinoid tumors, bone-associated cancers, leukemias, lymphomas (NHL) and the like.

A plurality of the oral dosage forms of the invention or prepared by the process of the invention can be given to a patient to provide a single total dosage from about 1 μg to about 500 mg, e.g., about 50 mg to 400 mg. The total dosage can be given in the form of a suitable number of the unit dosage forms, i.e., for a 200 mg dose of picoplatin, 4 unit dosage forms containing 50 mg each of picoplatin can be administered. Alternatively, a single or multiple unit dosage form can be given more frequently, for example, for one day up to daily for a period of weeks, e.g., for 1-10 weeks. It is preferred that the entire number of a plurality of the unit dosage form for a given administration be administered within a short interval of time, for example, within a period of time of about 5 minutes. Thus, if a given administration includes a nominal 200 mg of the picoplatin, and nominal dosage forms containing 50 mg each of picoplatin are used, all four capsules should be administered to the patient substantially concurrently. For dosage levels of about 0.001-400 mg total picoplatin, assuming a average patient body surface area of 1.7 m², these doses are equal to about 0.0006 to 235 mg/m², respectively. This dosage can be repeated as medically indicated; for example, in each treatment period, a single dose can be administered or the dosage can be repeated daily for a period of time, e.g., for up to ten weeks, with intervals between treatment periods, e.g., weekly intervals, about every two weeks, about every three weeks, about every four weeks, about every five weeks, or about every six weeks, as is deemed medically indicated. The dosing is preferably carried out so as to maintain an essentially constant therapeutic level of picoplatin in the treated subject for the desired period of time, e.g., for one day up to consecutive daily dosing for several weeks, i.e., for about 1-10 weeks.

The method of treatment of the invention can further include orally or parenterally administering, preferably sequentially (before or after) or concurrently (including simultaneously or overlapping), at least one additional medicament and/or anti-cancer therapy, such as radiation therapy, with a unit dosage form or a plurality of unit dosage forms comprising picoplatin, such as the unit dosage form(s) of the invention or prepared by the method of the invention. The additional medicament can be an anti-cancer medicament, preferably a non-Pt containing medicament, and may be administered orally or intravenously.

For example, an additional anti-cancer medicament can comprise, without limitation, a taxane (e.g., paclitaxel or docetaxel), a growth factor receptor inhibitor (e.g., an antibody such as bevacizumab or cetuximab or AZD2171 (Recentin)), an anti-metabolite (capecitabine, gemcitabine or 5-FU with or without leucovorin), a PK inhibitor (e.g., sorafenib tosylate), an anthracyclin (amrubicin, doxorubicin or liposomal doxorubicin), a vinca alkaloid or an alkylating agent, including melphalan and cyclophosphamide. Useful agents include the platinum and non-platinum anticancer drugs disclosed in U.S. patent application Ser. Nos. 10/276,503, filed Sep. 4, 2003; 11/982,841, filed Nov. 5, 2007; 11/935,979, filed Nov. 6, 2007; 11/982,839, filed Nov. 5, 2007; in U.S. Pat. Nos. 7,060,808 and 4,673,668; in PCT WO/98/45331 and WO/96/40210 and in Provisional Application Ser. No. 60/889,171, filed Feb. 9, 2007 and in the Martell et al., U.S. provisional application Ser. No. ______, filed Feb. 8, 2008, entitled “Use of Picoplatin and Bevacizumab to Treat Colorectal Cancer” (Atty. Docket No. 295.114PRV); Martell et al., U.S. provisional application Ser. No. ______, filed Feb. 8, 2008, entitled “Use of Picoplatin and Cetuximab to Treat Colorectal Cancer” (Atty. Docket No. 295.115PRV); Karlin et al., U.S. provisional application Ser. No. ______, filed Feb. 8, 2008, entitled “Picoplatin and Amrubicin to Treat Lung Cancer” (Atty. Docket No. 295.116PRV); Martell et al., U.S. provisional application Serial No.______, filed Feb. 8, 2008, entitled “Combination Chemotherapy Comprising Stabilized Intravenous Picoplatin” (Atty. Docket No. 295.120PRV) (all of which are incorporated by reference herein). Alternatively, the additional medicament is a non-platinum containing anti-cancer agent, can be selected to treat a complication of the cancer, or to provide relief to a subject from at least one symptom of the cancer.

Preferred anti-cancer medicaments are those that can be administered orally, in effective doses, such as those listed on Table 1, below.

TABLE 1
Orally Administrable Agents

Altretamine
Anagrelide
Anastrozole
(ZD1033)
Bexarotene
Bicalutamide
Capecitabine
Clodronic acid
Cytarabine
ocfosfate
Dasatinib
Dutasteride
Erlotinib
Exemestane
Fadrozole
Finasteride
Fludarabine
Gefitinib
GMDP
HMPL 002
Hydroxycarbamide
Ibandronic acid
Idarubicin
Imatinib
Lapatinib
Lenalidomide
Letrozole
Osaterone
Polysaccharide K
Prednimustine
S1
(Gimeracil/oteracil/tegafur)
Sobuzoxane
Sorafenib
Sunitinib
Tamibarotene
Tamoxifen
Tegafur/uracil
Temozolomide
Thalidomide
Topotecan
Toremifene
Treosulfan
Trilostane
Ubenimex
Vinorelbine
Vorinostat

Preferred agents are oral formulations of Altretamine (alkylating agent) (Hexalen®), Capecitabine (anti-metabolite) (Xeoda®), Dasafinib (TK inhibitor) (Spryce®), Erlotinib (EGF receptor antagonist) (Tarceva®), Gefitinib (EGF inhibitor) (Iressa®), Imatinib (TK inhibitor) (Gleevec®), Lapatinib (Tycerb®) (EGFR inhibitor, Her2 inhibitor), Lenalidomide (TNF antagonist) (Revlimid®), Sunitinib (TK inhibitor) (Sutent®), S-1 (anti-metabolite), Sorafenib (angiogenesis inhibitor), Tegafur/Uracil (anti-metabolite) (UFT), Temozolomide (alkylating agent) (Temodar®), Thalidomide (Thalomid) (angiogenesis inhibitor), Topotecan (Hycamptin®), Vinorelbine (Navelbine®), and Vorinostat (HDI) (Zolinza®).

The invention further provides a kit comprising packaging containing separately packaged, a sufficient number of the unit dosage forms of the capsules of the invention or capsules prepared according to the method of the invention to provide for a course of treatment. A kit can further include instructional materials, such as instructions directing the dose or frequency of administration. For example, a kit can comprise sufficient daily doses for a prolonged period, such as a week or a plurality of weeks, or can comprises multiple unit dosage forms for a single administration when the dose is to be repeated less frequently, such as a daily dose. The multiple unit dosage forms can be packaged separately, but in proximity, as in a blister pack. The kit can also include separately packaged, a plurality of unit dosage forms of the non-platinum containing anti-cancer agent, preferably oral unit dosage forms.

### Example 1

### Oral Bioavailability Study

The population to be enrolled will be subjects with advanced non-hematological malignancies for whom no standard therapy exists and for whom treatments with single agent picoplatin is appropriate. Subjects may have previously received a platinum agent and be considered “platinum refractory” (e.g., subjects with lung cancer, head and neck cancer, ovarian cancer or other malignancies often treated with platinum-based chemotherapy) or may not have received prior platinum-based chemotherapy (e.g., subjects with sarcomas, breast cancer, carcinoid tumors, etc).

The Study design is a randomized, two-period crossover, open label study in which a single dose (Cycle 1) of picoplatin is given either IV or PO, followed 4 weeks later by a single dose (Cycle 2) of picoplatin given by the route not used for Cycle 1.

The IV dose was 120 mg/m² administered over one hour. This dose is extrapolated from the maximum tolerated dose in heavily pre-treated subjects, likely to be characteristic of the patient population to be studied.

Oral dose levels studied sequentially (6 subjects per dose level) in the absence of dose limiting toxicity: 200 mg, 300 mg, or 400 mg total dose per subject. Assuming an average BSA of 1.7 m², these doses are equal to approximately mean doses of 119, 164, and 235 mg/m². Assuming a relative bioavailability of 50% for the oral formulation, these oral doses would be equal to approximately 60, 90, and 120 mg/m² given intravenously, or 52, 72 and 103 mg/m² if bioavailability is about 44%.

Blood and urine samples, taken at specified time points after each dose of study drug, are analyzed for total platinum concentrations in plasma (bound platinum) and plasma ultrafiltrate (unbound platinum).

Capsules, which are opaque white, are sealed with a green band. The appropriate number of capsules for the prescribed dose are removed from the bottle and placed in a drug envelope (or other vessel) such that the subject can easily slide the capsules into the mouth without touching the capsules. Protection from light while transporting capsules to the subjects for ingestion is preferred. The composition of a capsule containing 50 mg of picoplatin is given in Table 2, below.

TABLE 2
Composition of Picoplatin Gelatin Capsule 50 mg

| Ingredient | Amount (mg) | Function |
|---|---|---|
| Picoplatin | 50 | Active Ingredient |
| Lactose 450# | 116 | Filler |
| Avicel PH101 | 20 | Binder/Filler |
| Croscarmellose Sodium | 8 | Disintegrant |
| Polyvidone | 4 | Disintegrant/binder |
| Magnesium Sterate | 2 | Glidant |

Picoplatin capsules are taken orally with the subject swallowing the entire prescribed dose over 5 minutes with 240 mL (8 ounces) of water (after consumption of clear liquids and anti-emetic therapy only during the preceding 4 hours).

Premedication anti-emetics include dexamethasone, 8-12 mg, (or equivalent corticosteroid) and a 5-HT₃ receptor antagonist given PO or IV immediately prior to the study drug. Subjects also receive anti-emetic therapy as needed for several days following treatment, e.g., oral lorazepam, prochlorperazine, or equivalent as clinically indicated. Each subject must receive the same anti-emetic regimen (drugs, dose and route) during Cycles 1 and 2.

### Results

Plasma bioavailability of orally administered picoplatin at doses of 119 mg/m² and 164 mg/m² was respectively 39±15% and 28±16% (P═NS) measured as the AUC of plasma platinum and 44%±4% and 27%±10% (P=0.015) as the AUC of plasma ultrafiltrate platinum. An exposure saturation profile presented at these doses. Both bound and unbound platinum peak concentrations following picoplatin oral and IV dosing occurred at 3-5 hr and 1 hr (coincides with end of the IV infusion) respectively. Circulating plasma half-life is similar to that established after an intravenous infusion, ranging between 55 and 77 hours in plasma ultrafiltrate (PUF). Four weeks after picoplatin dosing by either route, only background levels of circulating platinum could be detected, suggesting no drug accumulation between dosing cycles. Comparable prolonged plasma platinum terminal half-life was observed with mean values established at 125±10 hr and 134±20 hr (P═NS) respectively after picoplatin dosing by IV and oral route. No differences were observed between pharmacokinetics of Cycle 1 and Cycle 2 dosing. All doses were well tolerated and no serious adverse events were noted following the oral dose. There was no evidence of myelosuppression after oral dosing.

### Example 2

### Projected Intermittent Dosing Schedules

Based on measured platinum exposure following oral dosing, several dosing scenarios can be employed, involving intermittent oral dosing. For example, picoplatin can be administered orally as a single agent once daily for 8 weeks in doses approaching and/or achieving the MTD. After 2 weeks off the drug, patients with no evidence of tumor progression or clinically limiting, significant toxicity would be permitted to repeat additional 8 week cycles of the same dose of picoplatin. The patient population to be studied would be similar to that to be studied in Example 1, as described above, i.e., patients with non-hematological malignancy for which there is no curative therapy and for which treatment with single agent picoplatin is appropriate. Examples can include patients with recurrent small or non-small cell lung cancer, head and neck cancer, pancreatic, cervical, prostate or ovarian cancer who are not candidates for or choose not to be treated with commercially available chemotherapeutic agents. The daily dose of oral picoplatin will be escalated in serial cohorts to establish the maximum tolerated doses (MTD).

When used in combination therapy picoplatin can be administered orally once daily with a second non-Pt anticancer agent, such as capecitabine in patients who are to receive capecitabine for any malignancy in which concurrent therapy with platinum chemotherapy is felt to be clinically appropriate, e.g., non-small cell lung cancer, head and neck cancer, pancreatic, cervical, anal or rectal cancer. The dose of the second agent, e.g., capecitabine to be used will be defined after review of relevant and timely data of single agent and low-dose combination clinical trials in the relevant cancer, such as colorectal or breast cancer in the case of capecitabine. In addition, the frequency (i.e., daily for 7 days and then 7 days off therapy or daily for 14 days and then 7 days off therapy) will also be determined by review of relevant and timely data at the time the study is to be initiated. The combination chemotherapy would be repeated until tumor progression or unacceptable toxicity. The initial daily dose of picoplatin will be 50% of the MTD determined in Example 1 and then will be escalated in serial cohorts to establish the MTD of oral picoplatin given daily in combination with the initial dose and schedule of the second agent, e.g., capecitabine. Depending on the adverse events observed and the MTD of picoplatin with the second agent at the initial dose and schedule studied, subsequent dose cohorts may seek to determine the MTD of picoplatin with alternative doses of the second agent, either higher or lower than the initial dose studied and/or with alternative schedules of on/off oral dosing of both agents.

Picoplatin also can be administered orally as a single agent once daily for 5-7 weeks combined with radiation therapy, e.g., about 180-200 cGy, 5 days/week, to total radiation dose of about 4500-6500 cGy over the 5-7 week course of treatment. This would be done in patients who are to receive radiation therapy for any malignancy in which concurrent therapy with platinum chemotherapy is felt to be clinically appropriate. This would include, for example, patients with good performance status but locally recurrent, symptomatic small or non-small cell lung cancer, head and neck cancer, pancreatic, cervical, breast, colon, prostate or ovarian cancer. The initial daily dose of picoplatin will be 50% of the MTD determined in Example 1, and then will be escalated in serial cohorts to establish the MTD of oral picoplatin given daily during radiation therapy. Picoplatin can be administered via the present capsules, or in coated tablets or pills, or via ingestion of liquid formulations.

A third intermittent dosing scenario would consist of daily administration of picoplatin doses up to about 350-450 mg each day for 3 consecutive days followed by recovery period of about 7 days. This dosing scenario can be used to quickly reach optimal therapeutic levels of picoplatin, such as by approaching or achieving maximal levels, e.g., as by saturating, the levels of picoplatin in the blood circulation, thus aggressively attacking the cancer cells. Due to the immunosuppressant side effects, the recovery drug-free period can assist the immune system recovery, as well as permitting substantial elimination, e.g., about 90-100% elimination of picoplatin from circulation prior to the next dosing cycle.

A different intermittent dosing scenario would be considered for patients able to tolerate a more continuous exposure to picoplatin. An example of such therapy would consist of daily oral dosing of picoplatin ranging between about 1 μg and 10 mg, up to about 4 continuous weeks (28 days), followed by a 1-3 weeks (e.g., about 14 days) picoplatin-free recovery period which would benefit the recovery of the immune system from the immunosuppressant effect imposed by picoplatin. Several dosing cycles can be used depending on patient disease status. This dosing scenario aims at maintaining steady levels of blood circulating picoplatin for a continuous exposure and effect on the cancer cells.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A unit dosage form for picoplatin, adapted for oral administration of the picoplatin, comprising a substantially water-soluble capsule shell, the capsule shell enclosing a formulation comprising a substantially dry powder comprising about 10 to 60 wt % particulate picoplatin, a substantially water-soluble, water-dispersible, or water-absorbing carbohydrate, and an effective amount of up to about 5 wt % of a lubricant.

2. The unit dosage form of claim 1 wherein the particulate picoplatin is of less than about 10 microns average particle diameter.

3. The unit dosage form of claim 2 wherein about 90% of the particulate picoplatin has a particle diameter of less than about 5 microns.

4. The unit dosage form of claim 1, 2 or 3 wherein the particulate picoplatin is micronized, microcrystalline, lyophilized, or any combination thereof.

5. The unit dosage form of claim 1 or 2 wherein the particulate picoplatin is dispersed within substantially every particle of the powder of the formulation.

6. The unit dosage form of claim 1 or 2 wherein the formulation does not comprise an oxidant, a metal oxide, or a compound comprising a halo, ═N(H), —NH₂, or —SH moiety.

7. The unit dosage form of claim 1 or 2 wherein the carbohydrate comprises a monosaccharide, a disaccharide, a sugar alcohol, a cellulose, a modified cellulose, or a mixture thereof.

8. The unit dosage form of claim 7 wherein the carbohydrate comprises lactose, sucrose, mannitol, sorbitol, microcrystalline cellulose, or a mixture thereof.

9. The unit dosage form of claim 1 or 2 wherein the capsule shell comprises hard gelatin, gelatin/PEG, or hydroxypropyl methyl cellulose.

10. The unit dosage form of claim 9 wherein the capsule shell is a two part shell that further comprises a capsule band covering the seam between the two parts.

11. The unit dosage form of claim 1 or 2 wherein the capsule shell is substantially light-attenuating.

12. The unit dosage form of claim 11 wherein the capsule shell is opaque.

13. The unit dosage form of claim 12 wherein the capsule shell comprises or is externally coated with an effective amount of an opaquifying agent.

14. The unit dosage form of claim 13 wherein the opaquifying agent is TiO₂.

15. The unit dosage form of claim 1 or 2 wherein the lubricant comprises an alkaline earth metal salt of a fatty acid.

16. The unit dosage form of claim 15 wherein the alkaline earth metal salt of a fatty acid is magnesium stearate.

17. The unit dosage form of claim 1 or 2 wherein the formulation further comprises about 5-10 wt % of a dispersant.

18. The unit dosage form of claim 17 wherein the dispersant comprises croscarmellose sodium.

19. The unit dosage form of claim 17 wherein the dispersant comprises polyvinylpyrrolidone.

20. The unit dosage form of claim 7 wherein the modified cellulose comprises a cellulose ether.

21. The unit dosage form of claim 20 wherein the cellulose ether is methyl cellulose, ethyl cellulose, hydroxyethyl cellulose, hydroxypropyl cellulose, hydroxypropyl methyl cellulose, or a combination thereof.

22. The unit dosage form of claim 1 or 2 wherein the carbohydrate comprises about 40-80 wt % of the formulation.

23. The unit dosage form of claim 7 wherein the modified cellulose is a microcrystalline cellulose.

24. The unit dosage form of claim 4 wherein the picoplatin particulate has been micronized by jet milling.

25. The unit dosage form of claim 9 comprising a substantially opaque, banded, capsule shell enclosing the formulation, the formulation comprising about 50 to 200 mg of picoplatin particulate, lactose, microcrystalline cellulose, magnesium stearate, and croscarmellose sodium or povidone or both.

26. A process for preparing an encapsulated unit dosage form for picoplatin, adapted for oral administration of the picoplatin, comprising preparing a formulation comprising a substantially dry powder comprising about 10 to 60 wt % particulate picoplatin, a substantially water-soluble, water-dispersible, or water-absorbing carbohydrate, and an effective amount of up to about 5 wt % of a lubricant; and enclosing the formulation within a substantially water-soluble capsule shell.

27. The process of claim 26 wherein the particulate picoplatin is of less than about 10 microns average particle diameter.

28. The process of claim 27 wherein about 90% of the particulate picoplatin has a particle diameter of less than about 5 microns.

29. The process of claim 26, wherein the picoplatin particulate is micronized, microcrystalline, or lyophilized, or any combination thereof.

30. The process of claim 26 or 27 wherein the particulate picoplatin is dispersed within substantially every particle of the powder of the formulation.

31. The process of claim 26 or 27 wherein the carbohydrate comprises a monosaccharide, a disaccharide, a sugar alcohol, a cellulose, a modified cellulose, or a mixture thereof.

32. The process of claim 26 or 27 wherein the carbohydrate comprises lactose, sucrose, mannitol, sorbitol, microcrystalline cellulose, or a mixture thereof.

33. The process of claim 26 or 27 wherein the lubricant comprises an alkaline earth metal salt of a fatty acid.

34. The process of claim 33 wherein the alkaline earth metal salt of a fatty acid is magnesium stearate.

35. The process of claim 29 wherein the micronized particulate picoplatin is produced by jet milling.

36. The process of any one of claim 26 or 27 wherein the capsule shell is a two part hard shell that further comprises a capsule band covering the seam between the two parts.

37. The process of claim 36 wherein the capsule shell comprises hydroxypropyl methyl cellulose.

38. The process of claim 36 wherein the capsule shell is a hard gelatin capsule.

39. The process of claim 36 wherein the capsule shell is light-attenuating.

40. The process of claim 39 wherein the capsule shell comprises or is coated with an effective amount of an opaquifying agent.

41. The process claim 40 wherein the capsule shell comprises or is coated with an effective opaquifying amount of TiO₂.

42. The process of any one of claim 26 or 27 wherein the formulation further comprises about 5-10 wt % of a dispersant.

43. The process of claim 42 wherein the dispersant comprises croscarmellose sodium.

44. The process of claim 42 wherein the dispersant comprises polyvinylpyrrolidone.

45. The process of claim 26 or 27 wherein the picoplatin is substantially anhydrous.

46. The process of claim 31 wherein the carbohydrate comprises a cellulose ether.

47. The process of claim 46 wherein the cellulose ether comprises methyl cellulose, ethyl cellulose, hydroxyethyl cellulose, hydroxypropyl cellulose, or hydroxypropyl methyl cellulose, or a mixture thereof.

48. The process of claim 26 or 27 wherein the carbohydrate comprises about 40-80 wt % of the composition.

49. The process of claim 31 wherein the modified cellulose comprises a microcrystalline cellulose.

50. The unit dosage form for picoplatin prepared by the process of claim 26 or 27.

51. The unit dosage form of claim 50 comprising about 0.001-400 mg of particulate picoplatin.

52. A method of treating cancer in a human afflicted therewith, comprising administering orally at least one unit dosage form of claim 1, at a total dose per administration, at a frequency, and over a period of time adequate to provide a beneficial effect.

53. A method of treating cancer comprising (a) consecutive oral daily administration of at least one unit dosage form comprising picoplatin to a human afflicted with cancer, so as to attain the optimal therapeutic level of picoplatin in the circulation of the human; and (b) discontinuing said administration for a period of time effective for the mammal to substantially eliminate said picoplatin from its circulation.

54. The method of claim 53 wherein the optimal therapeutic level is attained in about 3-5 days.

55. The method of claim 53 wherein the optimal therapeutic level is a saturation level of picoplatin in the circulation.

56. The method of treating cancer comprising (a) consecutive daily oral administration of said at least one unit dosage form comprising picoplatin to a human afflicted with cancer, so as to achieve a uniform sub-maximal level of said picoplatin in the circulation of the human; and (b) discontinuing said administration for a period of time effective for the human to substantially eliminate said picoplatin from its circulation.

57. The method of claim 56 wherein the daily administration is carried out at least once, for about 3-5 weeks.

58. The method of claim 57 wherein the administration is carried out for about 5-7 weeks.

59. The method of claim 57 wherein the administration is carried out for up to about 1-2 years.

60. The method of claim 59 wherein, in step (b), the period of time is no more than about 2-3 weeks.

61. The method of claim 56 wherein the administration is of 10% or less of the maximum tolerated dose.

62. The method of claim 53 or 56 wherein about 10-50% of the picoplatin within the unit dosage form is bioavailable to the human being after oral ingestion.

63. The method of claim 53 or 56 further comprising repeating steps (a) and (b) a plurality of times.

64. The method of claim 53 or 56 further comprising orally administering at least one non-platinum anti-cancer agent to the human sequentially or concurrently with the picoplatin.

65. A kit comprising packaging containing, separately packaged, a sufficient number of the unit dosage forms of claim 1 to provide for a course of treatment for a mammal afflicted with cancer.

66. The kit of claim 56 comprising instructional materials directing the dose and/or frequency of administration.

67. The kit of claim 66 further comprising, separately packaged, a plurality of oral unit dosage forms of a non-platinum anti-cancer agent.
